SEQUENCE LISTING (19 sequences)

SEQ ID NO: 1 - protein, 20 aa, Foot-and-mouth disease virus
NAVPNLRGDLQVLAQKVART

SEQ ID NO: 2 - protein, 14 aa, Foot and mouth disease virus
RGDLQVLAQKVART

SEQ ID NO: 3 - protein, 14 aa, Artificial Sequence
  Synthetic peptide
ILNMRTDLGTLLFR

SEQ ID NO: 4 - protein, 10 aa, Artificial Sequence
  Synthetic peptide
RTDLGTLLFR

SEQ ID NO: 5 - protein, 12 aa, Artificial Sequence
  Synthetic peptide
RTDLDSLRTYTL

SEQ ID NO: 6 - protein, 8 aa, Artificial Sequence
  Synthetic peptide
RSDLTPLF

SEQ ID NO: 7 - protein, 14 aa, Foot and mouth disease virus
RGDLQVLAQKVART

SEQ ID NO: 8 - protein, 14 aa, FOOT AND MOUTH DISEASE VIRUS
RGDRQVLAQKVART

SEQ ID NO: 9 - protein, 14 aa, FOOT AND MOUTH DISEASE VIRUS
RGDLAVLAQKVART

SEQ ID NO: 10 - protein, 14 aa, FOOT AND MOUTH DISEASE VIRUS
RGDLQVAAQKVART

SEQ ID NO: 11 - protein, 14 aa, FOOT AND MOUTH DISEASE VIRUS
RGDLQVLAQKAART

SEQ ID NO: 12 - protein, 16 aa, FOOT AND MOUTH DISEASE VIRUS
NLRGDLQVLAQKVART

SEQ ID NO: 13 - DNA, 39 bp, Artificial Sequence
  Primer
attgatgaattcctccatggcttttcccccgcggcgacg

SEQ ID NO: 14 - DNA, 39 bp, Artificial Sequence
  Primer
gacatgctagcggccgcattaagtttctgagtttccttc

SEQ ID NO: 15 - DNA, 44 bp, Artificial Sequence
  Primer
cactatgaattccgtctcacatggggattgaactgctttgcctg

SEQ ID NO: 16 - DNA, 43 bp, Artificial Sequence
  Primer
tcatacactagtgcggccgcctagcaatctgtggaaaggtcta

SEQ ID NO: 17 - protein, 18 aa, Homo sapiens
MLLVTSLLCELPHPAFLL

SEQ ID NO: 18 - protein, 9 aa, Artificial Sequence
  Synthesized linker sequence
GGGGSGGGS

SEQ ID NO: 19 - protein, 18 aa, Artificial Sequence
  Synthesized linker sequence
GSTSGSGKPGSGEGSTKG

(19) United States
(12) Patent Application Publication
(10) Pub. No.: US 2017/0233452 A1
(43) Pub. Date: Aug. 17, 2017

(54) CHIMERIC ANTIGEN RECEPTORS SPECIFIC TO AVB6 INTEGRIN AND METHODS OF USE THEREOF TO TREAT CANCER

(71) Applicant: DISCOVERY GENOMICS, INC., Minneapolis, MN (US)

(72) Inventors: R. Scott MCIVOR, St. Louis Park, MN (US); Kendra A. HYLAND, Little Canada, MN (US)

(73) Assignee: Immusoft Corporation, Seattle, WA (US)

(21) Appl. No.: 15/306,055

(22) PCT Filed: Apr. 23, 2015
(86) PCT No.: PCT/US15/27332

§ 371 Date: Oct. 21, 2016

Related U.S. Application Data
(60) Provisional application No. 61/983,082, filed on Apr. 23, 2014.

(51) Int. Cl. C07K 14/705; A61K 39/00; C07K 14/725

(52) U.S. Cl. CPC C07K 14/70546; C07K 14/7051; C07K 14/70521; A61K 39/0011; C07K 2319/03; C07K 2319/30; A61K 2039/5158

(57) ABSTRACT

Disclosed are chimeric antigen receptors (CAR) specific to αvβ6 integrin which is uniquely expressed in a wide variety of cancers. Also disclosed are vectors to express the CAR and methods to use the CAR to treat patients suffering from cancer. The instant disclosure provides a CAR comprising a binding domain specific to αvβ6 integrin. In various exemplary embodiments, the αvβ6 specific binding domain comprises a sequence as defined by SEQ ID NOs. 1-12. In some embodiments, the CAR comprises one or more intracellular domains comprising 4-1 BB domain, CD3ζ domain, and CD28 domain. In some embodiments, the αvβ6 binding domain is fused to an Fc region by a glycine-serine linker. In these and other embodiments, the Fc region is substantially similar to an lgG4 or an lgG1 Fc region.

### CROSS REFERENCE TO RELATED APPLICATIONS

This application claims priority to U.S. Provisional Application No. 61/983,082 entitled “CHIMERIC ANTIGEN RECEPTORS BINDING ALPHA V BETA 6” filed Apr. 23, 2014, the contents of which are hereby incorporated herein in their entirety for all purposes.

### STATEMENT OF GOVERNMENT SUPPORT

Aspects of the work described herein were supported by National Institutes of Health Grant-1 R43 CA176957-01. The United States Government may have certain rights in this invention.

### FIELD OF THE INVENTION

The invention relates generally to novel chimeric antigen receptors with binding specificity to αvβ6 integrin which is displayed on cell surfaces. In adults, αvβ6 integrin is, generally, uniquely displayed on cancer cells providing a vehicle and method to specifically target cancer cells for therapy.

### BACKGROUND

Pancreatic cancer represents the 10th most common cancer diagnosis, yet the 4th most common estimated cause of death. The only potential curative therapy for pancreatic cancer is surgical resection; however, few patients have tumors that can be resected. Pancreatic ductal adenocarcinoma, which represents 90% of pancreatic cancers, is particularly aggressive, since it rapidly metastasizes and often expresses growth factors and signaling components that permit rapid growth. Alternative therapies are desperately needed, as there have been no recent medical advances for treatment of pancreatic adenocarcinoma.

### INTRODUCTION TO THE INVENTION

There are many treatments and proposed treatments for cancer or pancreatic cancer. One general area of cancer research involves immunotherapy by adoptive transfer of engineered T cells that are intended to mediate cancer regression and overcome evasive mechanisms by which tumors avoid immune responses. There are various techniques of T-cell modification. One technique for modifying T-cells is to create chimeric antigen receptors (CAR) that are introduced into the T-cells. CAR are engineered fusion molecules comprising an antigen-binding motif and intracellular signaling domains. CAR can recognize tumor antigens independently of major histocompatiblity complex (MHC), expression of which is often lost by tumor cells.

There are many cancer treatment proposals directed to treating cancer based on antigens expressed by the cancer cells. Finding a suitable target antigen, however, is difficult or even impossible. Cells naturally have many antigens and many of the same antigens. A cancer cell might not have any unique antigens, or there might not be any antigens unique to a cancer that is shared by enough of the cancer patient population to make developing a treatment possible. For example, others have made major research investments in the prostate stem cell antigen (PSCA), and have made and tested CARs directed to PSCA¹. This research contributes to scientific progress but it now appears that PSCA may be shared by various normal tissues and might not be a suitable target antigen.

Therefore, there is a need for identification of specific epitopes that are unique to cancer cells and was to target them that result in death of the cancer cell.

### SUMMARY OF THE INVENTION

Disclosed are chimeric antigen receptors (CAR) specific to αvβ6 integrin which is uniquely expressed in a wide variety of cancers. Also disclosed are vectors to express the CAR and methods to use the CAR to treat patients suffering from cancer.

The instant disclosure provides a CAR comprising a binding domain specific to αvβ6 integrin. In various exemplary embodiments, the αvβ6 specific binding domain comprises a sequence as defined by SEQ. ID NOs. 1-12 (Table 1 and 2). In some embodiments, the CAR comprises one or more intracellular domains comprising 4-1BB domain, CD3 domain, and CD28 domain. In these embodiments, the intracellular domains may be disposed in any possible order, with any one of the domains being on the COOH terminus of the CAR and the other domain or domains being adjacent to the same. In various exemplary embodiments, the CAR comprises a transmembrane domain comprising a CD4 or a CD8 transmembrane domain or a portion thereof. In some embodiments, the αvβ6 binding domain is fused to an Fc region by a glycine-serine linker. In these and other embodiments, the Fc region is substantially similar to an IgG₄ or an IgG₁ Fc region.

In other exemplary embodiments, the disclosure provides a cell that expresses a CAR comprising a binding domain specific to αvβ6 integrin as disclosed in the preceding paragraph. In some embodiments, the cell is an immune cell. In various embodiments the immune cell is a T cell or a natural killer cell. In some embodiments, the immune cell is a human immune cell. In some aspects, the binding of the CAR results in interferon-γ secretion. In some embodiments, activation of the T cell results in death of a cell expressing the αvβ6 integrin. In various embodiments the cell expressing the αvβ6 integrin is a cancer cell. In these and other embodiments, the cancer cell is a pancreatic cancer cell, a colon cancer cell, an ovarian cancer cell, a breast cancer cell, oral cancer cell, skin cancer cell, stomach cancer cell, basal cell, liver cell, gastric, cervical squamous or an endometrium cancer cell.

In other exemplary embodiments, disclosed are vectors suitable for the expression of a CAR comprising a binding domain specific to αvβ6 integrin, as disclosed in the preceding paragraphs. In these and other embodiments, the CAR is expressed from a plasmid or is integrated into and expressed from genomic DNA. In some exemplary embodiments, the vector includes a transposase.

In yet other exemplary embodiments, disclosed are a nucleic acid for expression of a CAR comprising: a) a nucleic acid sequence encoding a binding domain, the binding domain having specific binding to αvβ6 integrin; b) a nucleic acid sequence encoding a transmembrane domain, and c) a nucleic acid sequence encoding an intracellular signaling domain. In some embodiments, the nucleic acid further comprises a sequence encoding an Fc region of an antibody. In various embodiments, the nucleic acid also includes a dimerizable antibody hinge portion. In still other embodiments the nucleic acid comprises a flexible linker. In some embodiments, the binding domain is an antibody, an antibody fragment or a peptide ligand for αvβ6 integrin. In some embodiments the αvβ6 integrin binding domain comprises SEQ. ID. NOs. 1-12 (Tables 1 and 2).

In yet other exemplary embodiments the invention provides a vector comprising the nucleic acid of any embodiment of the preceding paragraphs. In these embodiments, the vector may also include and transposon or a transposase or an integrating viral vector. In some exemplary embodiments, the invention provides a template for homologous recombination for a nucleic acid of the preceding paragraphs. In these and other embodiments, the invention, the nucleic acid, vector or template may comprise DNA, cDNA, RNA or mRNA.

In yet other exemplary embodiments, disclosed is a method of treating a patient in need thereof comprising administering a CAR with binding specificity to αvβ6 integrin as disclosed in any of the preceding paragraphs. In these embodiments, administering comprises preparing a cell to express the CAR and administering the cell to the patient. In some exemplary embodiments, the cell is a T-cell or an NK cell. In various embodiments, the cell is a human cell. In various exemplary embodiments, the treatment is for cancer. In these embodiments, the cancer is endometrial, basal cell, liver, colon, gastric, cervical squamous, oral, pancreas, breast and ovary. In some embodiments, the cells are taken from the patient, prepared ex vivo to express the CAR and then administered or reintroduced to the patient.

These and other features and advantages of the inventions will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be apparent from the description, as set forth hereinafter.

### BRIEF DESCRIPTION OF THE FIGURES

Various exemplary embodiments of the compositions and methods according to the invention will be described in detail, with reference to the following figures wherein:

FIGS. 1A and 1B: The design of the CAR Transposons and structure. FIG. 1A, Design of Sleeping Beauty T2 transposons encoding anti-αvβ6-binding domain, fused to IgG4 hinge by Gly-Ser linker, lacking or containing intracellular signaling domains from CD28, 4-1BB and CD3ζ. FIG. 1B, Structure of CAR when expressed on the cell surface, with 4-1BB and CD3ζ intracellular signaling domains.

FIGS. 2A and 2B: A single repeat of the A14 or A20 binding domain from FMDV2 VP1 protein exhibited the greatest binding to soluble αvβ6 integrin. FIG. 2A, A20 cells (a mouse B cell line) were electroporated with transposons encoding CAR containing one repeat of the indicated binding domains (Table 1). One day post electroporation, CAR expression was detected by staining with a goat anti-human IgG Alexa Fluor 647 conjugated antibody. Integrin αvβ6 binding was detected using anti-αv PE monoclonal antibody (clone NKI-M9). Data are expressed as percentage of CAR+ cells. A14 CAR did not bind to soluble αvβ3 integrin. The percentage of CAR+(hIgG+) cells binding to αvβ6 integrin is presented. FIG. 2B, Mouse B cell line (A20), expressing CAR encoding a single A14 domain, bound more frequently to soluble αvβ6 integrin (5 μM) compared to CAR containing duplicate A14 domains, linked with glycine-serine (GS) or ARL linker. Cells were stained as described in (A) and analyzed by flow cytometry. The horizontal dashed line indicates the percentage of cells binding to anti-αv antibody in the absence of αvβ6 integrin.

FIG. 3: Primary T cells subsets (CD3+CD4+ or CD3+CD8+) express CAR encoding αvβ6 binding domain (A14) after nucleofection with SB transposon. Peripheral blood mononuclear cells were nucleofected (Amaxa Nucleofector) with SB transposon plasmids encoding A14 CAR and different combinations of intracellular signaling domains (FIG. 1). One day post nucleofection, cells were stained for CD3ε, CD4, CD8 and anti-hIgG (CAR) and analyzed by flow cytometry

FIG. 4: Soluble αvβ6 binding to CAR. Constructs having the binding domain whons in Table 2 bind soluble αvβ6 integrin protein. Data provided shows % αvβ6+, % of CAR+.

FIG. 5: Primary T cells stably express CAR in a donor dependent manner. Results are presented as percent of CAR+/CD3+ expressing cells of the total of mature CD3+ T cells.

FIGS. 6A and 6B: T cells expressing A14-41BB-CD3z CAR secrete interferon-γ (IFNγ) after exposure to αvβ6 protein (FIG. 6A) or αvβ6+ pancreatic cancer cells (BxPC-3) (FIG. 6B).

FIG. 7: T cells expressing CAR with signaling domains 41BB-CD3z CAR kill CFSE+αvβ6+ pancreatic cancer cells (Capan 2).

FIGS. 8A and 8B: Pancreatic cancer cell lines express αvβ6 integrin. FIG. 8A. The fluorescent signal from AsPC-1 stained with 10D5 (grey) overlapped with that of the isotype control antibody. FIG. 8B. Relative expression of αvβ6 integrin by Capon? cells and K562 αvβ6+ clones (artificial antigen presenting cells). Expression of αvβ6 was detected after staining with antibody clone 10D5 or isotype control mouse IgG2b, followed by goat anti-mouse IgG Alexa Fluor 647.

### DETAILED DESCRIPTION OF THE EXEMPLARY EMBODIMENTS

Unless defined otherwise, all technical and scientific terms used herein have the same meanings as commonly understood by one of ordinary skill in the art to which this invention belongs. All publications and patents specifically mentioned herein are incorporated by reference for all purposes including describing and disclosing the chemicals, instruments, statistical analyses and methodologies which are reported in the publications which might be used in connection with the invention. All references cited in this specification are incorporated herein by reference. Nothing herein is to be construed as an admission that the invention is not entitled to antedate such disclosure by virtue of prior invention.

The present disclosure is directed to research on the ability of several antigen-binding domains in the context of CAR to bind αVβ6, an integrin that is highly expressed on pancreatic cancer cells. A CAR incorporating a peptide from foot-and-mouth disease virus VP1 capsid protein, a human IgG Fc spacer region, a transmembrane spanning domain and a fusion of intracellular signaling domains from CD28 and CD3zeta provided the highest level of binding to αVβ6. Primary T-cells were then engineered for CAR expression using the Sleeping Beauty transposon system. Artificial antigen presenting cells expressing αVβ6 integrin protein were engineered for specific expansion of CAR-expressing primary T cells. Functional activation of the assembled CAR in primary T-cells was determined by secretion of IFNγ upon exposure of the engineered T cells to either αVβ6 protein or αVβ6+ pancreatic cancer cells. These results demonstrate the feasibility of assembling CAR to target αVβ6 integrin and support anticipated antitumor activity of these cells in preclinical studies and ultimately in the treatment of human pancreatic cancer.

Chimeric antigen receptors (CAR) are engineered molecules, consisting of an extracellular antigen-binding motif fused to intracellular signaling domains, which permit cellular activation upon ligand binding. Unlike endogenous T cell receptors, which bind to antigens in the context of MHC molecules, CAR have the advantage of recognizing tumor antigens in the absence of antigen processing pathways and MHC expression². Importantly, CARs do not have to be matched to the patient's MHC and can recognize tumor that has reduced expression of MHC. Target antigens of CARs currently are limited to cell surface proteins, reviewed recently by³.

The majority of tumors do not express any co-stimulatory molecules, and therefore co-stimulatory domains must be incorporated into the CAR molecule for efficient T cell activation. Early versions of CARs (“first generation”) contained a binding domain, typically an antibody-derived single chain variable region (scFv) and the intracellular signaling domain from CD3, which mediates antigen-specific cytotoxic activity and IL-2 production in murine T-cell hybridomas⁴. CD3ζ is an intracellular component of the T cell receptor complex that transmits signal to activate T cells. Expression of the combination of a scFv and CD3 chain was not sufficient to activate resting T cells from transgenic mice⁵. “Second generation” CARs have incorporated a co-stimulatory domain in addition to CD3 activation domain. The addition of the signaling domain from CD28 augments the ability of receptors to stimulate cytokine secretion and enhance antitumor activity in animal models^{6,7}. In addition, the CD28 costimulatory domain enhances the resistance of CAR+ T cells to regulatory T cells⁸ and improves in vivo persistence in human patients compared to CARs encoding only the CD3 activation domain⁹. CD137 (4-1BB) protein, a member of the TNF receptor family, is expressed by T cells after antigen-receptor signaling occurs, and can mediate survival signaling by T cells¹⁰. The combination of 4-1BB and CD3 intracellular signaling domains with either an anti-CD19 or anti-mesothelin scFv demonstrated extended T cell persistence in mouse xenograft models compared to the combination of CD28 and CD3ζ signaling domains^{11,12}. Recent clinical trials have demonstrated that the combination of 4-1BB and CD3 intracellular signaling domains with anti-CD19 CAR mediates T cell persistence and have resulted in dramatically positive patient outcomes^{13,14}.

Integrins are alpha-beta heterodimeric glycoproteins that mediate adhesion between cells and act as a bridge between cells and the extracellular matrix^{15,16}. Integrins additionally serve as a bidirectional interface between the cell and its environment to regulate signal transduction, cellular differentiation, migration, and proliferation¹⁷⁻²⁰. Integrins in the alpha V family, including αvβ1, αvβ3, αvβ5, αvβ6, and αvβ8 bind their ligands after recognition of a highly conserved arginine-glycine-aspartic acid (RGD) motif. Integrin αvβ6 binds to extracellular matrix proteins, including fibronectin, tenascin, and vitronectin²¹⁻²³. In addition, alphaV family integrins, including αvβ6, bind to the precursors of TGFβ1 and TGFβ3 and mediate cleavage of latency-associated peptide (LAP) from functional TGFβ^{21,24,25}. αvβ6 is expressed by epithelial cells during development and wound healing, but expression is low or absent in adult tissue²⁶.

Integrin αVβ6 constitutes a potentially effective target for T cell-based cancer therapy. Integrin αvβ6 is overexpressed by several types of carcinomas, including gastric carcinoma, lung adenocarcinoma, ovarian carcinoma and pancreatic adenocarcinomas^{27,28}. In a histological survey of adenocarcinomas of gastroenteropancreatic origin, αVβ6 expression was strongest in pancreatic ductal carcinoma, compared to esophageal, gastric, and colon carcinoma²⁷. In addition, αVβ6 is over-expressed by epithelial ovarian tumors²⁸, head and neck squamous cell carcinomas²⁹, and non small cell lung cancer³⁰. Other studies using immunohistochemistry and immunoprecipitation have identified αVβ6 expression in cancer of the endometrium, basal cell, liver, colon, gastric, cervical squamous cell, oral SCC, pancreas, breast and ovary⁴⁹.

Foot and mouth disease virus (FMDV) recognizes αVβ6 as its primary receptor³¹. A component of the FMDV capsid, VP1 protein, contains a flexible loop (GH loop), including an RGD motif that mediates binding to αVβ6. FMDV can also binding to αvβ3 and αvβ8. A 20-mer peptide derived from FMDV serotype O₁ BFS (Table 1, A20-FMDV2) mediated the strongest binding to αVβ6 compared to peptides derived from other FMDV serotypes or the latency associated peptide of TGFβ1³². In addition, peptides that bind αvβ6 integrin containing an RTD motif have been identified from synthetic libraries (Table 1)^{33,34}. Anti-αvβ6 binding domains have been used in studies for in vivo imaging of αVβ6⁺ cancers, including pancreatic cancer³⁵⁻³⁹.

TABLE 1

| | | | | SEQ |
|---|---|---|---|---|
| | | | | ID |
| NAME | SEQUENCE | SOURCE | REFERENCE | NO. |
| A20- | NAVPNLRGDLQVLAQKVART | GH loop | DiCara | 1 |
| FMDV2 | | of VP1 | et al. | |
| | | protein | 2007 J Biol | |
| | | of FMDV | Chem. | |
| | | sero- | 282(13): | |
| | | types | 9657 | |
| | | O1 BFS | | |
| A14- | RGDLQVLAQKVART | GH loop | DiCara | 2 |
| FMDV2 | | of VP1 | et al. | |
| | | protein | 2007 J Biol | |
| | | of FMDV | Chem. | |
| | | sero- | 282(13): | |
| | | types | 9657 | |
| | | O1 BFS | | |
| R01- | ILNMRTDLGTLLFR | Synthe- | Kimura | 3 |
| 14 | | tic | et al. | |
| | | peptide | Clin Can | |
| | | library | Res 2011. | |
| | | | 18(3): 839 | |
| R01- | RTDLGTLLFR | Synthe- | Kimura | 4 |
| 10 | | tic | et al. | |
| | | peptide | Clin Can | |
| | | library | Res 2011. | |
| | | | 18(3): 839 | |
| Bpep | RTDLDSLRTYTL | Phage | Kraft | 5 |
| | | library | et al. | |
| | | | J Biol | |
| | | | Chem. | |
| | | | 1999. | |
| | | | 274(4): | |
| | | | 1979 | |
| TopPep | RSDLTPLF | Synthe- | Gagnon | 6 |
| #1-8 | | tic | et al. | |
| | | peptide | PNAS. | |
| | | library | 2009. | |
| | | | 106(42): | |
| | | | 17904 | |

The inventors have created synthetic receptors (“chimeric antigen receptor” or CAR) containing anti-αvβ6 binding domains from FMDV or synthetic peptide libraries which binds to carcinoma cells expressing αvβ6 integrin.

### Materials and Methods

### Sleeping Beauty Transposons

Transposons were constructed using T2 inverted terminal repeat sequences as described⁴⁰, separated by 1,800 base pairs (bp) of bacterial sequence consisting of the ColE1 bacterial origin of replication and kanamycin (Kan) resistance gene. The CLP promoter transcriptionally regulates CAR expression, which contains a CpG-less promoter element derived from pCpG-free-mcs (Life Technologies, San Diego, Calif.), consisting of murine CMV enhancer, CpG-free elongation factor 1-α (EF1α) promoter and intron sequences⁴¹. The rabbit beta globin polyadenylation signal in the pKT2/ZOG transposon⁴¹ was replaced with the bovine growth hormone polyadenylation signal (BGH pA), by digesting plasmid pcDNA3.1⁽⁺⁾ (Life Technologies) with NotI and NheI and ligating to create pKT2/CLP-BGH pA transposon.

### Transposase Plasmids

Both pCMV-SB11⁴² and pCMV-SB100x⁴³ were used in the described experiments.

### CAR-Sequence Assembly

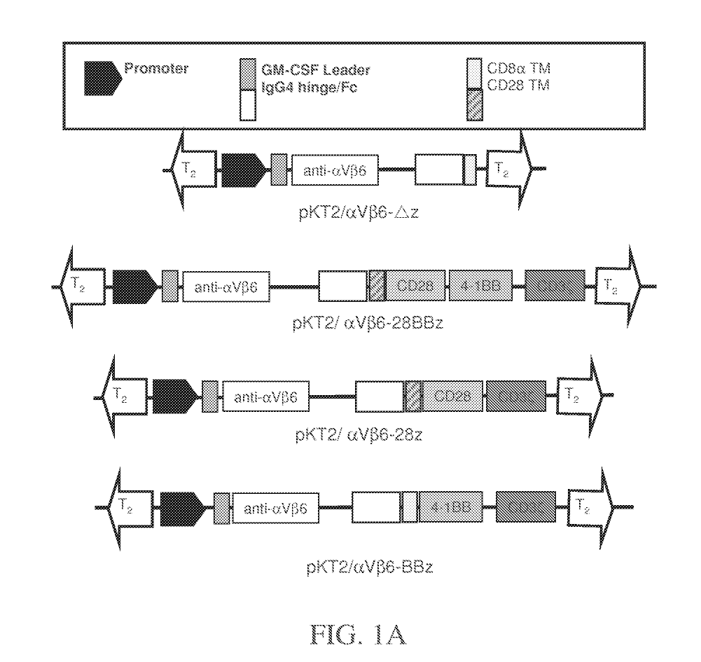

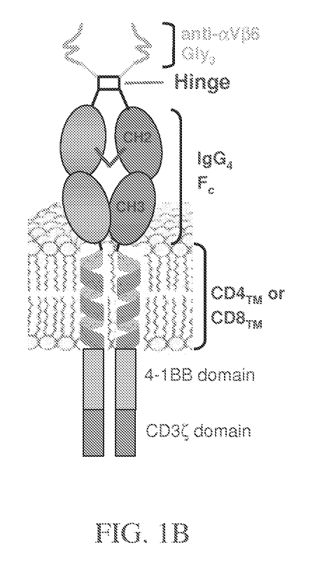

The CAR DNA sequence (FIG. 1A) encodes a GM-CSF receptor alpha leader sequence (60 bp, MLLVTSLLCELPHPAFLL) (SEQ ID NO 17), anti-αvβ6 binding domain (24-60 bp, Table 1), a triple glycine linker (27 bp, GGGGSGGGS) (SEQ ID NO 18), human IgG4 corresponding to the Fc and hinge domains (684 bp, corresponding to amino acids 99 to 327, GenBank P01861), and the CD4 transmembrane domain (TM) (66 bp, from amino acids 219-240, NP_001181943) (FIG. 1A). The sequence was human codon optimized, substituting codons with those optimally used in mammals without altering the anticipated amino acid sequence, and synthesized by DNA 2.0 (Menlo Park, Calif.). The IgG4 hinge contains two point mutations: 1) substitution of proline for serine at residue 109 in the hinge region to stabilize disulfide bonds between the heavy chains; 2) substitution of glutamic acid for leucine at residue 116 in the CH2 region to reduce binding to FcγRI and activation of macrophages, monocytes and natural killer cells⁴⁴. The CAR sequence was digested with BsmBI and NotI and ligated into transposon pKT2/CLP-BGH pA between NcoI to NotI sites to generate pKT2/CLP-CARBGH polyA.

In one CAR transposon, double repeats of the A14 binding domain (Table 1) were fused by either the glycine-serine (GGGGSGGGS) (SEQ ID NO 18) or ARL linkers (GSTSGSGKPGSGEGSTKG)⁴⁵.

### Intracellular Signaling Domains

In pKT2/anti-αvβ6-BBz, the CD4 TM domain was replaced by the CD8a TM domain (84 bp, amino acids 183-209, GenBank NP_001759.3), and fused to CD137/4-1BB (141 bp, amino acids 208 to 255, GenBank NP_006130.1). This sequence was in turn fused to the cytoplasmic portion of the human CD247/CD3 molecule (339 bp, amino acids 51 to 164, GenBank NP_932170.1). In pKT2/anti-αvβ6-28z, the CD4 TM was replaced with the extracellular, transmembrane and cytoplasmic portions of human CD28, from amino acids IEVMY to the C-terminus (123 bp, GenBank NP_006130.1)⁶, including a modification to remove a dileucine motif (RLLH-->RGGH⁴⁶) at amino acids 186 to 187. In anti-αvβ6-28BBz, the CD28 domain is fused in frame to the 4-1BB domain and the CD3ζ domain (FIG. 1A).

### Artificial Antigen Presenting Cells Expressing αvβ6

### Integrin-Encoding Transposon Construction

Transposons contained a bi-directional promoter⁴⁷, derived from pKT2-SE⁴⁰, with the CLP promoter transcriptionally regulating integrin expression. Drug-resistance genes puromycin-N-acetyltransferase or neomycin phosphotransferase were transcriptionally regulated by the PGK (phosphoglycerate kinase) promoter. Human integrin alpha V isoform 1 (ITGAV, Gene ID 3685, IMAGE clone BC126231) was PCR amplified using forward primer: 5′-attgatgaattcctccatggcttttcccccgcggcgacg-3′ (SEQ ID NO 13) and reverse primer: 5′-gacatgctagcggccgcattaagtttctgagtttccttc-3′ (restriction sites are underlined) (SEQ ID NO 14), digested with NcoI and NotI, then ligated into pKT2/CLP-PGK-neomycin to create pKT2/ITGAV-CLP-PGK-neomycin transposon. Human beta 6 integrin isoform A (ITGB6, GeneID 3693, IMAGE clone BC121178) was PCR amplified using forward primer: 5′-cactatgaattccgtctcacatggggattgaactgctttgcctg-3′ (SEQ ID NO 15) and reverse primer 5′-tcatacactagtgcggccgcctagcaatctgtggaaaggtcta-3′ (restriction sites are underlined) (SEQ ID NO 16), digested with NcoI and NotI, then ligated into pKT2-CLP-PGK-Puro to create pKT2/ITGB6-CLP-PGK-Puro transposon.

### K562 Genetic Modification and Cloning

K562 cells were electroporated using the Nucleofector I system (Lonza, Walkersville). One million cells in 100 μl of Ingenio buffer (Minis) were electroporated using the Amaxa Nucleofector I program T-16 (Lonza) with a total of 7 μg transposon (1:3 molecular ratio of pKT2/ITGAV-CLP-PGK-neomycin to pKT2/ITGB6-CLP-PGK-Puromycin) and 2 μg of pCMV-SB11 (2:1 Tn:Ts ratio)⁴². Two days post electroporation, cells were plated with 1.2 μg/ml G418 and 2 μg/ml puromycin (Calbiochem). Heterodimer expression of αvβ6 was determined by staining with mouse anti-αvβ6 antibody (clone 10D5, Millipore²³), followed by goat anti-mouse-IgG-AlexaFluor 647 (R&D Systems), and flow cytometry (LSRII, BD Biosciences). K562 cells were cultured for 12 d providing fresh medium and selective agents three times weekly and then plated in methylcellulose (HSC002; R&D Systems, Minneapolis, Minn.) containing G418 and puromycin. After incubating for 12 days, colonies were picked and screened for expression by flow cytometry with anti-αvβ6 antibody (clone 10D5, Millipore). The K562 clone exhibiting the highest αvβ6 expression (#3-5) was identified, expanded and cryopreserved. These cells were then used for antigen presentation after irradiation using an X-ray irradiator (100 Gy, Rad Source Technologies).

CAR Binding to αvβ6 Integrin Assay.

Both A20 and Jurkat cell lines (ATCC) were cultured in RPMI-1640 with 10% FBS. A20 cells (2 million) were electroporated in 200 μl of BTXpress buffer (Harvard Apparatus) using 2 mm cuvettes and a Cytopulse Electroporator, with 2 pulses at 900V/cm for 5 msec (Cytopulse). Jurkat cells (2 million) were mixed with 15 μg plasmid DNA and electroporated in 200 μl Ingenio buffer (Minis) using an Amaxa Nucleofector I, program D-23 (Lonza). One day post electroporation, CAR expressing cells were washed in integrin binding buffer (25 mM Tris, pH7.4, 150 mM NaCl, 1 mM MgCl₂, 2 mM CaCl₂, 1 mM MnCl₂, 1% BSA (Fraction V), 0.1% NaN₃) and incubated with varying concentrations of recombinant soluble αVβ6 (R&D Systems), followed by phycoerythrin (PE) conjugated-anti-αV antibody, (clone NKI-M9, Biolegend), and F(ab)₂ antibody fragment goat anti-human IgG (Fcγ specific), conjugated to Alexa Fluor 647 (Jackson Immunoresearch). Cells were analyzed by flow cytometry (FACsCalibur, BD).

### Results

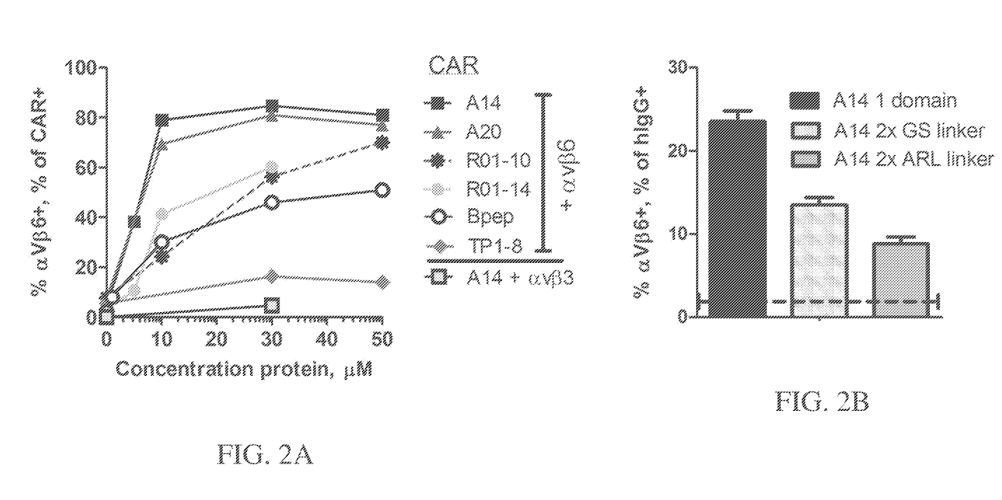

Sleeping Beauty transposons encoding CAR were assembled with an anti-αvβ6 binding domain fused by glycine-serine linker to the hinge and Fc fragment of human IgG4 and transmembrane domain from CD4 but lacking any intracellular signaling domains (FIGS. 1A and 1B). The binding domains have been characterized as binding αvβ6 integrin in different protein contexts, and used for imaging purposes^{32-34,38,48}. The ability of the assembled CAR containing a single repeat of an anti-αvβ6 domains (Table 1) to bind soluble αvβ6 integrin was assessed by flow cytometry after electroporation of a mouse B-cell line (A20 cells) with CAR-encoding transposon. CAR encoding the A14 and A20 binding domains from FMDV exhibited the highest level of binding to soluble αvβ6 integrin at the lowest αvβ6 integrin concentration assayed (5-10 μM) (FIG. 2A). The TopPep#1-8 sequence did not bind soluble αvβ6 in this context. CAR containing A14 domain did not bind to soluble αvβ3, demonstrating CAR specificity (FIG. 2A). A doublet repeat of the A14 domain linked by flexible linkers exhibited reduced ability of CAR expressing cells to bind to soluble αvβ6 protein (FIG. 2B).

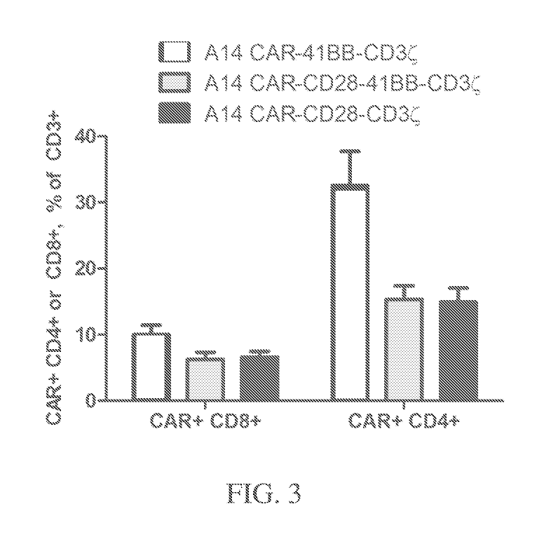

A CAR encoding the A14 binding domain was assembled with CD3C intracellular signaling domain in combination with 4-1BB and/or CD28 signaling domains (FIG. 1A). After nucleofection of human peripheral blood mononuclear cells with CAR transposon, both CD3+CD4+ and CD3+CD8+ T cells expressed all versions of CAR on their surface (FIG. 3).

TABLE 2

| | | | SEQ |
|---|---|---|---|
| | | | ID |
| NAME | BINDING DOMAIN | REFERENCE | NO. |
| A14-FMDV2 | RGDLQVLAQKVART | DiCara 2007 | 7 |
| A14-FMDV2-L4R | RGDRQVLAQKVART | Burman | 8 |
| | | et al 2006 | |
| A14-FMDV2-Q5A | RGDLAVLAQKVART | Burman | 9 |
| | | et al 2006 | |
| A14-FMDV2-L7A | RGDLQVAAQKVART | DiCara 2007 | 10 |
| A14-FMDV2-V11A | RGDLQVLAQKAART | DiCara 2007 | 11 |
| A16-FMDV2 | NLRGDLQVLAQKVART | Burman | 12 |
| | | et al 2006 | |

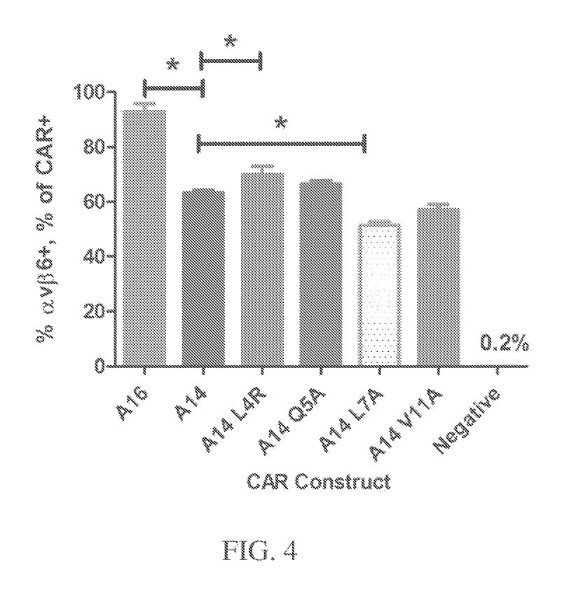

CAR containing the A16 sequence from FMDV2 bound the best to soluble αvβ6 integrin protein (FIG. 4). A20 cells were electroporated with transposons encoding CAR containing one repeat of the indicated binding domains (Table 2). One day post electroporation, CAR expression was detected by staining with a goat anti-human IgG Alexa Fluor 647 conjugated antibody. Integrin αvβ6 binding was detected using anti-αv PE monoclonal antibody (clone NKI-M9). Data are expressed as percentage of CAR+ cells. The percentage of CAR+(hIgG+) cells binding to αvβ6 integrin is presented.

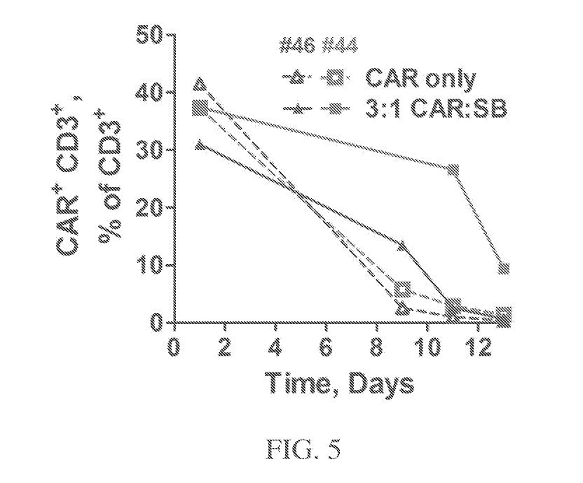

Primary T cells stably express CAR in a donor dependent manner (FIG. 5). Peripheral blood mononuclear cells from two different donors (#46 and #44) were nucleofected (Amaxa Nucleofector I, U14 program) with SB transposon plasmids encoding A14-41BB-CD3z CAR. A separate treatment group were nucleofected with the SB A14-41BB-CD3z CAR as well as pCMV-SB100× at a ratio of 1:3 and cultured in OpTmizer T cell expansion serum free media. One day post nucleofection, anti-CD3/CD28 Dynabeads were added with 50 IU/ml IL-2 to further stimulate T cells. On day 13 post nucleofection, cells were stained for CD3ε, CD4, CD8 and anti-hIgG (CAR), and CAR+ cells were 90% CD8+ and 10% CD4+. Results are presented as percent of CAR+/CD3+ expressing cells of the total of mature CD3+ T cells.

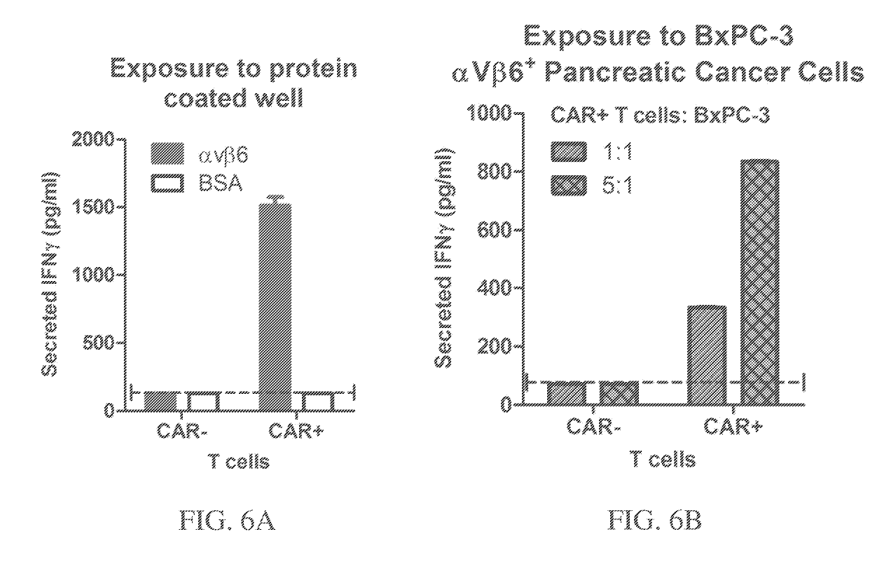

T cells expressing A14-41BB-CD3z CAR secrete interferon-γ (IFNγ) after exposure to αvβ6 protein or αvβ6+ pancreatic cancer cells (BxPC-3) (FIGS. 6A and 6B). Peripheral blood mononuclear cells were activated for 6 d by culture with beads coated with anti-CD3/anti-CD28 antibodies (Life Technologies). Beads were removed, and cells were nucleofected (Amaxa Nucleofector) with SB transposon plasmid. One day post nucleofection, CAR+ T cells were plated with BxPC-3 cells (1×10⁴) or in plates coated with αvβ6 protein. Cell free media were collected 2 days later and assayed for IFN γ by ELISA (R&D Systems). As illustrated, CAR+ T cells secreted IFNγ in response to either BxPC-3 or in response to pancreatic cancer cells (BxPC). BxPC-3 had the highest level of αvβ6 integrin expression amongst four pancreatic cancer cell lines, while AsPC-1 had little or no surface expression of αvβ6 (Data not shown).

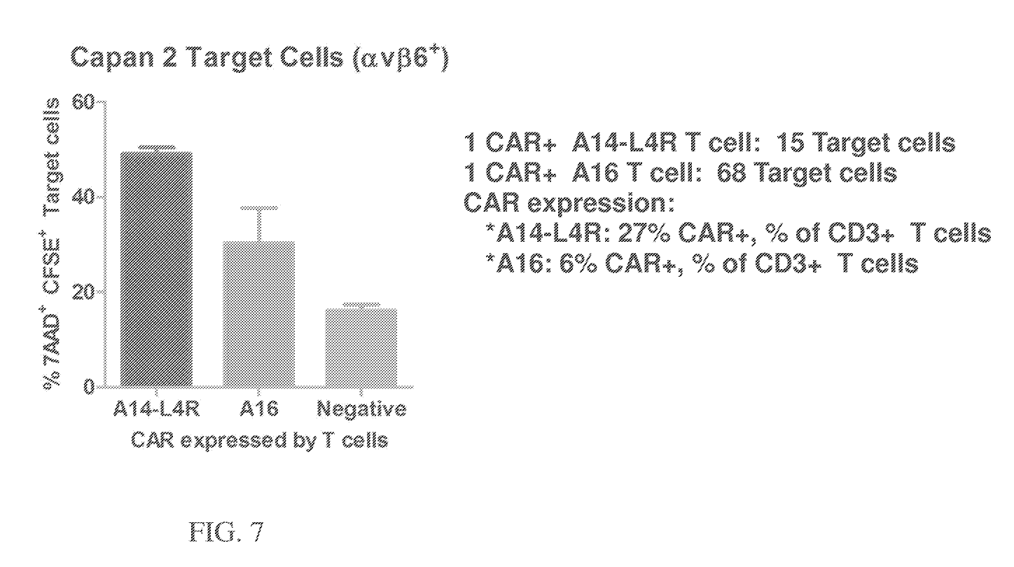

Human T cells expressing CAR (A14-L4R or A16) kill Capan 2 pancreatic cancer cells (FIG. 7). T cells expressing CAR with signaling domains 41BB-CD3z CAR kill pancreatic cancer cells (Capan 2) expressing αvβ6+ as measured by carboxyfluorescein succinimidyl ester (CFSE) staining. Peripheral blood mononuclear cells were activated for 7 d by culture with beads coated with anti-CD3/anti-CD28 antibodies (Life Technologies). The beads were removed, and cells were nucleofected (Amaxa Nucleofector) with SB transposon plasmid encoding CAR or not. One day post nucleofection, T cells (5×10⁴) were plated with Capan-2 cells (1×10⁴) for 4 h, followed by staining with 7AAD and analysis by flow cytometry. Samples were run in duplicate.

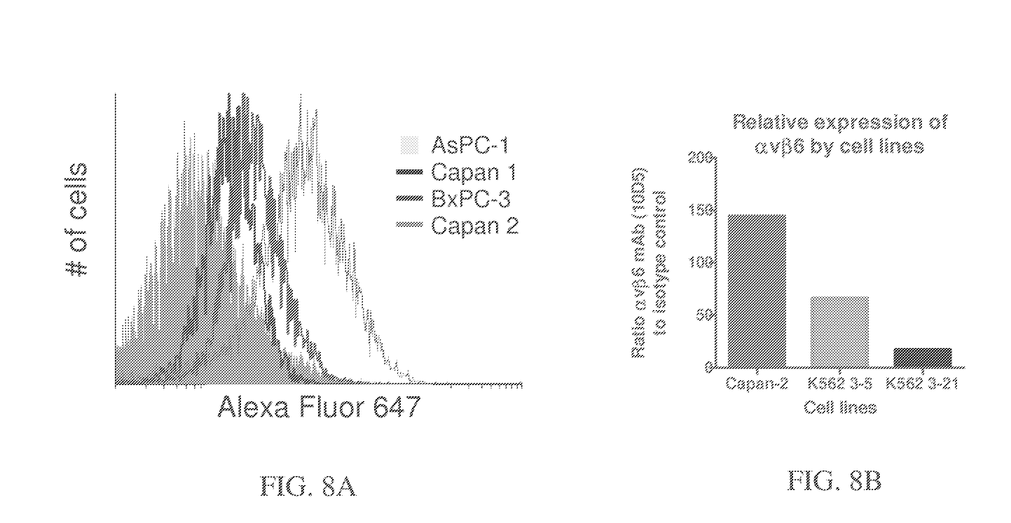

To confirm the ubiquity of the αvβ6 integrin in pancreatic cancer, four different pancreatic cell lines were labeled with a fluorescent antibody to the αvβ6 integrin. These results FIG. 8A confirm AsPC-1, Capan 1, BxPC-3 and Capan 2 express αvβ6 integrin. The fluorescent signal from AsPC-1 stained with 10D5 (grey) overlapped with that of the isotype control antibody. These results were confirmed by comparing pancreatic cell line to K562 cells artificially expressing αvβ6 integrin (FIG. 8B). Relative expression of αvβ6 integrin by Capan2 cells and K562 αvβ6+ clones. Expression of αvβ6 was detected after staining with antibody clone 10D5 or isotype control mouse IgG2b, followed by goat anti-mouse IgG Alexa Fluor 647.

These results have demonstrated that the combination of an anti-αvβ6 binding domain in the context of a CAR molecule can bind to αvβ6 integrin. In addition, the assembled CAR can mediate antigen-specific T cell activation when exposed to target carcinoma cells expressing αvβ6. Further, the results provided herein show that T cell activation mediated by the CAR can result in cell death providing a vehicle to specifically target cancer cell to cause cell death.

Certain embodiments of the invention include, for example, all domains which bound to αvβ6 protein as set forth herein, including conservative substitutions to the same. These include all the sequences in Table 1, except for TopPep#1-8, which did not bind αvβ6. Certain embodiments of the invention include all combinations of the intracellular signaling domains that were assembled (see FIG. 1A). Inventions include CAR expressing T cells, the cells being effective in mediating cytotoxicity against αvβ6 expressing pancreatic tumor cells both in vitro and/or in vivo. Certain embodiments of the invention include use of these cells for anti-tumor therapeutics against pancreatic or other αvβ6 expressing tumors.

### Vectors and Nucleic Acids

T-cells and other cells may receive vectors to express CARs and genetic constructs as described herein.

A variety of nucleic acids may be introduced into cells. As used herein, the term nucleic acid includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. The deoxyribose phosphate backbone can be modified to produce morpholino nucleic acids. In addition, the deoxyphosphate backbone can be replaced with, for example, a phosphorothioate or phosphorodithioate backbone, a phosphoroamidite, or an alkyl phosphotriester backbone. A nucleic acid sequence can be operably linked to a regulatory region such as a promoter for expression. As used herein, operably linked refers to positioning of a regulatory region relative to a nucleic acid sequence in such a way as to permit or facilitate transcription of the target nucleic acid. Any type of promoter can be operably linked to a target nucleic acid sequence. Examples of promoters include, without limitation, tissue-specific promoters, constitutive promoters, and promoters responsive or unresponsive to a particular stimulus.

Nucleic acid constructs can be introduced into embryonic, fetal, or adult cells of any type, including, for example, cells of the immune system, T-cells, antigen presenting cells, lymphocytes using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells. In transposon systems, the transcriptional unit of a nucleic acid construct, i.e., the regulatory region operably linked to a target nucleic acid sequence, is flanked by an inverted repeat of a transposon. Several transposon systems, including, for example, Sleeping Beauty (see, U.S. Pat. No. 6,613,752 and U.S. Publication No. 2005/0003542); Frog Prince (Miskey et al. (2003) Nucleic Acids Res. 31:6873); Tol2 (Kawakami (2007) Genome Biology 8(Suppl.1):57; Minos (Pavlopoulos et al. (2007) Genome Biology 8(Suppl.1):S2); Hsmar1 (Miskey et al. (2007) Mol Cell Biol. 27:4589); and Passport have been developed to introduce nucleic acids into cells, including mice, human, and pig cells. The Sleeping Beauty and Passport transposon is particularly useful. A transposase can be delivered as a protein, encoded on the same nucleic acid construct as the target nucleic acid, can be introduced on a separate nucleic acid construct, or provided as an mRNA (e.g., an in vitro-transcribed and capped mRNA).

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, and also any necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., HIV-1, SIV or FIV), retrovirus (e.g., ASV, ALV or MoMLV), and transposons (e.g., Sleeping Beauty, P-elements, Tol-2, Frog Prince, piggyBac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand).

The nucleic acid sequences set forth herein are intended to represent both DNA and RNA sequences, according to the conventional practice of allowing the abbreviation “T” stand for “T” or for “U”, as the case may be, for DNA or RNA. Polynucleotides are nucleic acid molecules of at least three nucleotide subunits. Polynucleotide analogues or polynucleic acids are chemically modified polynucleotides or polynucleic acids. In some embodiments, polynucleotide analogues can be generated by replacing portions of the sugar-phosphate backbone of a polynucleotide with alternative functional groups. Morpholino-modified polynucleotides, referred to herein as “morpholinos,” are polynucleotide analogues in which the bases are linked by a morpholino-phosphorodiamidate backbone (see, e.g., U.S. Pat. Nos. 5,142,047 and 5,185,444). In addition to morpholinos, other examples of polynucleotide analogues include analogues in which the bases are linked by a polyvinyl backbone, peptide nucleic acids (PNAs) in which the bases are linked by amide bonds formed by pseudopeptide 2-aminoethyl-glycine groups, analogues in which the nucleoside subunits are linked by methylphosphonate groups, analogues in which the phosphate residues linking nucleoside subunits are replaced by phosphoroamidate groups, and phosphorothioated DNAs, analogues containing sugar moieties that have 2′ O-methyl group). Polynucleotides can be produced through the well-known and routinely used technique of solid phase synthesis. Alternatively, other suitable methods for such synthesis can be used (e.g., common molecular cloning and chemical nucleic acid synthesis techniques). Similar techniques also can be used to prepare polynucleotide analogues such as morpholinos or phosphorothioate derivatives. In addition, polynucleotides and polynucleotide analogues can be obtained commercially. For oligonucleotides, examples of pharmaceutically acceptable compositions are salts that include, e.g., (a) salts formed with cations such as sodium, potassium, ammonium, etc.; (b) acid addition salts formed with inorganic acids, for example, hydrochloric acid, hydrobromic acid (c) salts formed with organic acids e.g., for example, acetic acid, oxalic acid, tartaric acid; and (d) salts formed from elemental anions e.g., chlorine, bromine, and iodine.

A sequence alignment is a way of arranging the sequences of DNA, RNA, or protein to identify regions of similarity. Aligned sequences of nucleotide or amino acid residues are typically represented as rows within a matrix, with gaps are inserted between the residues so that identical or similar characters are aligned in successive columns.

### Administration

The cells, including T-cells and other cells, may be modified to express CARs and administered to patients in a variety of ways. Autogenic cells taken from the patient are preferred, but cells from other sources may be used. One method involves collecting the cells from blood of the patient, modifying the cells ex vivo, and re-introducing them into the patient, e.g., by injection. Various molecules can be inserted into cells: vectors, drugs, DNA, proteins, or other molecules. In vivo modification of T-cells is also contemplated. mRNA and/or plasmids and/or vectors to express some or all of the CARs can be introduced into the cell ex vivo or in vivo. A patient may be treated one or more times.

One process of ex vivo modification includes electroporation. Electroporation is a technology that has been used in research laboratories throughout the world for the past 20 years. The primary application has been in transfection of eukaryotic and prokaryotic cells. The process subjects cells to a pulsed electric field for a short duration, resulting in permeabilization of the lipid bilayer of the cell membrane. This permeability develops in microseconds and resolves in seconds to minutes. While a physical “pore” has been observed under some circumstances, in most situations the permeability change is probably related to transient reorientation of membrane phospholipids. During the permeable period, both polar and non-polar molecules of various sizes can diffuse through the permeable areas according to concentration gradients. In addition, the electric field provides a force by which charged particles move into the cell (“electrophoretic” mechanism).

Examples of re-introduction into the patient includes via injection, such as intravenously, intramuscularly, or subcutaneously, and in/with a pharmaceutically acceptable carriers, e.g., in solution and sterile vehicles, such as physiological buffers (e.g., saline solution or glucose serum).

The following paragraphs enumerated consecutively from 1 through 38 provide for various embodiments:

1. A chimeric antigen receptor (CAR) comprising a binding domain specific to αvβ6 integrin.

2. The CAR of paragraph 1, wherein the αvβ6 integrin binding domain comprises SEQ. ID NO. 1, SEQ. ID NO 2, SEQ. ID NO 3, SEQ. ID NO 4, SEQ. ID NO 5, SEQ. ID NO 7, SEQ. ID NO 8, SEQ. ID NO 9, SEQ. ID NO 10, SEQ. ID NO 11 and SEQ. ID NO 12.

3. The CAR of paragraphs 1 and 2 comprising, in any combination, one or more intracellular domains comprising 4-1BB domain, CD3ζ domain, and CD28 domain.

4. The CAR of paragraphs 1-3, wherein the intracellular domains may be disposed in any possible order, with any one of the domains being on the COOH terminus of the CAR and the other domain or domains being adjacent to the same.

5. The CAR of paragraphs 1-4, further comprising a transmembrane domain.

6. The CAR of paragraphs 1-5, wherein the transmembrane domain is a CD4 or a CD8 transmembrane domain or a portion thereof.

7. The CAR of paragraphs 1-6, wherein the αvβ6 binding domain is fused to an Fc region by a glycine-serine linker.

8. The CAR of paragraphs 1-7, wherein the Fc region is substantially similar to an IgG₄ or an IgG₁ Fc region.

9. A cell or the CAR of paragraphs 1-8, wherein the CAR is expressed by a cell.

10. A cell or the CAR of paragraphs 1-9, wherein the cell is a human cell.

11. A cell or the CAR of paragraphs 1-10, wherein the cell is an immune cell.

12. A cell or the CAR of paragraphs 1-11, wherein the cell is a T cell or a natural killer (NK) cell.

13. A cell or the CAR of paragraphs 1-12, wherein binding of the CAR to an αvβ6 integrin expressing cell results in T cell or NK cell activation.

14. A cell or the CAR of paragraphs 1-13, wherein binding of the CAR results in interferon-γ secretion.

15. A cell or the CAR of paragraphs 1-14, wherein activation of the T cell results in death of a cell expressing the αvβ6 integrin.

16. A cell or the CAR of paragraphs 1-15, wherein the cell expressing the αvβ6 integrin is a cancer cell.

17. A cell or the CAR of paragraphs 1-16, wherein the cancer cell is a pancreatic cancer cell, a colon cancer cell, an ovarian cancer cell, a breast cancer cell, oral cancer cell, skin cancer cell, stomach cancer cell, basal cell, liver cell, gastric, cervical squamous or an endometrium cancer cell.

18. A vector suitable for the expression of a CAR according to any of paragraphs 1-17.

19. The vector according to any of paragraphs 1-18, wherein the CAR is expressed from a plasmid or is integrated into and expressed from genomic DNA.

20. The vector of any of paragraphs 1-19, wherein the vector comprises a transposase.

21. A nucleic acid for expression of a chimeric antigen receptor (CAR) comprising:
a. a nucleic acid sequence encoding a binding domain, the binding domain having specific binding to αvβ6 integrin;
b. a nucleic acid sequence encoding a transmembrane domain; and
c. a nucleic acid sequence encoding an intracellular signaling domain.

22. The nucleic acid of paragraph 21, further comprising a sequence encoding an Fc region of an antibody.

23. The nucleic acid of paragraphs 21-22, further comprising a sequence encoding a dimerizable antibody hinge portion.

24. The nucleic acid of paragraphs 21-23, comprising encoding a flexible linker.

25. The nucleic acid of paragraphs 21-24, wherein the binding domain is an antibody, an antibody fragment, or a peptide ligand for αvβ6 integrin.

26. The nucleic acid of paragraphs 21-25 wherein the binding domain is comprised of one or more of a peptide ligand comprising SEQ ID. NOs. 1-5 and 7-12.

27. A vector comprising the nucleic acid of any of paragraphs 21-26.

28. The vector of any of paragraphs 21-27, comprising a promoter for expression of the nucleic acid.

29. The vector of any of paragraphs 21-28, comprising a transposon or a transposase or an integrating viral vector.

30. A template for homologous recombination comprising the nucleic acid of any of paragraphs 1-29.

31. The vector of paragraphs 21-30 comprising or being DNA, cDNA, RNA, or mRNA.

32. A method of treating a patient in need thereof comprising: administering a CAR according to any of paragraphs 1-31.

33. The method of paragraph 32, wherein administering comprises preparing a cell to express the CAR and administering the cell to the patient.

34. The method of paragraphs 32-33, wherein the cell is a T-cell or an NK cell.

35. The method of paragraphs 32-35, wherein the cell is a human cell.

36. The method according to paragraphs 32-35, wherein the treatment is for cancer.

37. The method of paragraphs 32-36, wherein the cancer is endometrial, basal cell, liver, colon, gastric, cervical squamous, oral, pancreas, breast and ovary.

38. The method according to paragraphs 32-36, wherein the cells are taken from the patient, prepared ex vivo to express the CAR and then administered to the patient.

All patents, publications, and journal articles set forth herein are hereby incorporated by reference herein; in case of conflict, the instant specification is controlling.

While this invention has been described in conjunction with the various exemplary embodiments outlined above, various alternatives, modifications, variations, improvements, and/or substantial equivalents, whether known or that are or may be presently unforeseen, may become apparent to those having at least ordinary skill in the art. Accordingly, the exemplary embodiments according to this invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents of these exemplary embodiments.

### REFERENCES

1. Abate-Daga D, Lagisetty K H, Tran E, et al. A novel chimeric antigen receptor against PSCA mediates tumor destruction in a humanized mouse model of pancreatic cancer. Hum. Gene Ther. (2014).
2. Sadelain M, Brentjens R, Riviere I. The promise and potential pitfalls of chimeric antigen receptors. Curr Opin Immunol., 21:215-223 (2009).
3. Sadelain M, Brentjens R, Rivière I. The Basic Principles of Chimeric Antigen Receptor Design. Cancer Discov., 3 (4):388-398 (2013).
4. Eshhar Z, Waks T, Gross G, Schindler D G. Specific activation and targeting of cytotoxic lymphocytes through chimeric single chains consisting of antibody-binding domains and the gamma or zeta subunits of the immunoglobulin and T-cell receptors. Proc. Natl. Acad. Sci. U.S.A., 90(2):720-724 (1993).
5. Brocker T, Karjalainen K. Signals through T cell receptor-zeta chain alone are insufficient to prime resting T lymphocytes. J Exp. Med., 181(5):1653-1659 (1995).
6. Maher J, Brentjens R J, Gunset G, Rivière I, Sadelain M. Human T-lymphocyte cytotoxicity and proliferation directed by a single chimeric TCRzeta/CD28 receptor. Nat. Biotechnol., 20(1):70 (2002).
7. Friedmann-Morvinski D, Bendavid A, Waks T, Schindler D, Eshhar Z. Redirected primary T cells harboring a chimeric receptor require costimulation for their antigen-specific activation. Blood, 105(8):3087-3093 (2005).
8. Loskog A, Giandomenico V, Rossig C, et al. Addition of the CD28 signaling domain to chimeric T-cell receptors enhances chimeric T-cell resistance to T regulatory cells. Leukemia, 20(10):1819-1828 (2006).
9. Savoldo B, Ramos C A, Liu E, et al. CD28 costimulation improves expansion and persistence of chimeric antigen receptor-modified T cells in lymphoma patients. J Clin Invest., 121(5):1822-1826 (2011).
10. Wang C, Lin G H Y, McPherson A J, Watts T H. Immune regulation by 4-1BB and 4-1BBL: complexities and challenges. Immunol. Rev., 229(1):192-215 (2009).
11. Milone M C, Fish J D, Carpenito C, et al. Chimeric receptors containing CD137 signal transduction domains mediate enhanced survival of T cells and increased antileukemic efficacy in vivo. Mol. Ther., 17(8):1453-1464 (2009).
12. Carpenito C, Milone M C, Hassan R, et al. Control of large, established tumor xenografts with genetically retargeted human T cells containing CD28 and CD137 domains. Proc Natl Acad Sci USA., 106(9):3360-3365 (2009).
13. Porter D L, Levine B L, Kalos M, Bagg A, June C H. Chimeric antigen receptor-modified T cells in chronic lymphoid leukemia. N. Engl. J. Med., 365(8):725-733 (2011).
14. Kalos M, Levine B L, Porter D L, et al. T cells with chimeric antigen receptors have potent antitumor effects and can establish memory in patients with advanced leukemia. Sci. Transl. Med., 3(95):95ra73 (2011).
15. Rüegg C, Mariotti A. Vascular integrins: pleiotropic adhesion and signaling molecules in vascular homeostasis and angiogenesis. Cell. Mol. Life Sci., 60(6):1135-1157 (2003).
16. Eliceiri B P, Cheresh D A. Adhesion events in angiogenesis. Curr. Opin. Cell Biol., 13(5):563-568 (2001).
17. Jacquemet G, Humphries M J, Caswell P T. Role of adhesion receptor trafficking in 3D cell migration. Curr. Opin. Cell Biol., 25(5):627-632 (2013).
18. Geiger B, Yamada K M. Molecular architecture and function of matrix adhesions. Cold Spring Harb. Perspect. Biol., 3(5) (2011).
19. Bosman F T. Integrins: cell adhesives and modulators of cell function. Histochem. J., 25(7):469-477 (1993).
20. Springer T A. Adhesion receptors of the immune system. Nature, 346(6283):425-434 (1990).
21. Busk M, Pytela R, Sheppard D. Characterization of the integrin alpha v beta 6 as a fibronectin-binding protein. J. Biol. Chem., 267(9):5790-5796 (1992).
22. Prieto A L, Edelman G M, Crossin K L. Multiple integrins mediate cell attachment to cytotactin/tenascin. Proc. Natl. Acad. Sci. U.S.A, 90(21):10154-10158 (1993).
23. Huang X, Wu J, Spong S, Sheppard D. The integrin alphavbeta6 is critical for keratinocyte migration on both its known ligand, fibronectin, and on vitronectin. J Cell Sci., 111 (Pt 1:2189-2195) (1998).
24. Munger J S, Huang X, Kawakatsu H, et al. The integrin alpha v beta 6 binds and activates latent TGF beta 1: a mechanism for regulating pulmonary inflammation and fibrosis. Cell, 96(3):319-328 (1999).
25. Shi M, Zhu J, Wang R, et al. Latent TGF-β structure and activation. Nature, 474(7351):343-349 (2011).
26. Breuss J M, Gallo J, DeLisser H M, et al. Expression of the beta 6 integrin subunit in development, neoplasia and tissue repair suggests a role in epithelial remodeling. J Cell Sci., 108 (Pt 6:2241-2251) (1995).
27. Sipos B, Hahn D, Carceller A, et al. Immunohistochemical screening for beta6-integrin subunit expression in adenocarcinomas using a novel monoclonal antibody reveals strong upregulation in pancreatic ductal adenocarcinomas in vivo and in vitro. Histopathology, 45(3):226-236 (2004).
28. Ahmed N, Riley C, Rice G E, Quinn M A, Baker M S. Alpha(v)beta(6) integrin-A marker for the malignant potential of epithelial ovarian cancer. J Histochem. Cytochem., 50(10):1371-1380 (2002).
29. Hsiao J-R, Chang Y, Chen Y-L, et al. Cyclic alphavbeta6-targeting peptide selected from biopanning with clinical potential for head and neck squamous cell carcinoma. Head Neck, 32(2):160-172 (2010).
30. Elayadi A N, Samli K N, Prudkin L, et al. A peptide selected by biopanning identifies the integrin alphavbeta6 as a prognostic biomarker for nonsmall cell lung cancer. Cancer Res., 67(12):5889-5895 (2007).
31. Jackson T, Sheppard D, Denyer M, Blakemore W, King A M. The epithelial integrin alphavbeta6 is a receptor for foot-and-mouth disease virus. J Virol., 74(11):4949-4956 (2000).
32. DiCara D, Rapisarda C, Sutcliffe J L, et al. Structure-function analysis of Arg-Gly-Asp helix motifs in alpha v beta 6 integrin ligands. J. Biol. Chem., 282(13):9657-9665 (2007).
33. Kraft S, Diefenbach B, Mehta R, et al. Definition of an unexpected ligand recognition motif for alpha beta6 integrin. J. Biol. Chem., 274(4):1979-1985 (1999).
34. Kimura R H, Teed R, Hackel B J, et al. Pharmacokinetically Stabilized Cystine Knot Peptides that Bind Alpha-v-Beta-6 Integrin with Single-Digit Nanomolar Affinities for Detection of Pancreatic Cancer. Clin. cancer Res., 18(3):839-849 (2011).
35. Kogelberg H, Miranda E, Burnet J, et al. Generation and characterization of a diabody targeting the αvβ6 integrin. PLoS One., 8(9):e73260 (2013).
36. Saha A, Ellison D, Thomas G J, et al. High-resolution in vivo imaging of breast cancer by targeting the pro-invasive integrin alphavbeta6. J. Pathol., 222(1):52-63 (2010).
37. Hausner S H, Abbey C K, Bold R J, et al. Targeted in vivo imaging of integrin alphavbeta6 with an improved radiotracer and its relevance in a pancreatic tumor model. Cancer Res., 69(14):5843-5850 (2009).
38. Gagnon M K J, Hausner S H, Marik J, et al. High-throughput in vivo screening of targeted molecular imaging agents. Proc. Natl. Acad. Sci. U.S.A, 106(42):17904-17909 (2009).
39. Hausner S H, DiCara D, Marik J, Marshall J F, Sutcliffe J L. Use of a peptide derived from foot-and-mouth disease virus for the noninvasive imaging of human cancer: generation and evaluation of 4-[18F]fluorobenzoyl A20FMDV2 for in vivo imaging of integrin alphavbeta6 expression with positron emission tomography. Cancer Res., 67(16):7833-7840 (2007).
40. Cui Z, Geurts A M, Liu G, Kaufman C D, Hackett P B. Structure-function analysis of the inverted terminal repeats of the sleeping beauty transposon. J Mol Biol., 318(5):1221-1235 (2002).
41. Wilber A, Linehan J L, Tian X, et al. Efficient and stable transgene expression in human embryonic stem cells using transposon-mediated gene transfer. Stem Cells, 25(11):2919-2927 (2007).
42. Geurts A M, Yang Y, Clark K J, et al. Gene transfer into genomes of human cells by the Sleeping Beauty transposon system. Mol. Ther., 8(1):108-117 (2003).
43. Mátés L, Chuah M K L, Belay E, et al. Molecular evolution of a novel hyperactive Sleeping Beauty transposase enables robust stable gene transfer in vertebrates. Nat. Genet., 41(6):753-761 (2009).
44. Reddy M P, Kinney C a, Chaikin M a, et al. Elimination of Fc receptor-dependent effector functions of a modified IgG4 monoclonal antibody to human CD4. J. Immunol., 164(4):1925-1933 (2000).
45. Whitlow M, Bell B A, Feng S L, et al. An improved linker for single-chain Fv with reduced aggregation and enhanced proteolytic stability. Protein Eng., 6(8):989-995 (1993).
46. Nguyen P, Moisini I, Geiger TL. Identification of a murine CD28 dileucine motif that suppresses single-chain chimeric T-cell receptor expression and function. Blood, 102(13):4320-4325 (2003).
47. Multhaup M, Karlen A D, Swanson D L, et al. Cytotoxicity associated with artemis overexpression after lentiviral vector-mediated gene transfer. Hum. Gene Ther., 21(7):865-875 (2010).
48. Pameijer C R J, Navanjo A, Meechoovet B, et al. Conversion of a tumor-binding peptide identified by phage display to a functional chimeric T cell antigen receptor. Cancer Gene Ther., 14(1):91-97 (2007).
49. Bandyopadhyay A, Raghavan S. Defining the role of integrin alphavbeta6 in cancer. Curr Drug Targets, 10(7):645-52 (July 2009).

1. A chimeric antigen receptor (CAR) comprising a binding domain specific to αvβ6 integrin.

2. The CAR of claim 1, wherein the αvβ6 integrin binding domain comprises one or more of SEQ. ID NO. 1, SEQ. ID NO 2, SEQ. ID NO 3, SEQ. ID NO 4, SEQ. ID NO 5, SEQ. ID NO 7, SEQ. ID NO 8, SEQ. ID NO 9, SEQ. ID NO 10, SEQ. ID NO 11 and SEQ. ID NO 12.

3. The CAR of claim 1 comprising, in any combination, one or more intracellular domains comprising 4-1BB domain, CD3ζ domain, and CD28 domain.

4. The CAR of claim 3, wherein the intracellular domains may be disposed in any possible order, with any one of the domains being on the COOH terminus of the CAR and the other domain or domains being adjacent to the same.

5. The CAR of claim 1 further comprising a transmembrane domain.

6. The CAR of claim 5, wherein the transmembrane domain is a CD4 or a CD8 transmembrane domain or a portion thereof.

7. The CAR of claim 1 wherein the αvβ6 binding domain is fused to an Fc region by a glycine-serine linker.

8. The CAR of claim 7, wherein the Fc region is substantially similar to an IgG4 or an IgG1 Fc region.

9. A cell that expresses the CAR of claim 1.

10. (canceled)

11. (canceled)

12. The cell of claim 9, wherein the cell is a T cell or a natural killer (NK) cell.

13.-17. (canceled)

18. A vector suitable for expression of a CAR according to claim 1.

19. (canceled)

20. (canceled)

21. A nucleic acid for expression of a chimeric antigen receptor (CAR) comprising:
a. a nucleic acid sequence encoding a binding domain, the binding domain having specific binding to αvβ6 integrin;
b. a nucleic acid sequence encoding a transmembrane domain; and
c. a nucleic acid sequence encoding an intracellular signaling domain.

22.-24. (canceled)

25. The nucleic acid of claim 21, wherein the binding domain is an antibody, an antibody fragment, or a peptide ligand.

26. The nucleic acid of claim 25 wherein the binding domain is a peptide ligand comprising SEQ ID. NOs. 1-5 and 7-12.

27. A template for homologous recombination comprising the nucleic acid of claim 21.

28. A vector comprising the nucleic acid of claim 21.

29.-31. (canceled)

32. A method of treating a patient in need thereof comprising: administering a CAR according to claim 1.

33. The method of claim 32, wherein administering comprises preparing a cell to express the CAR and administering the cell to the patient.

34. (canceled)

35. (canceled)

36. The method according to claim 32, wherein the treatment is for cancer.

37. The method of claim 36, wherein the cancer is endometrial, basal cell, liver, colon, gastric, cervical squamous, oral, pancreas, breast and ovary.

38.-40. (canceled)
